(19) United States
(12) Patent Application Publication
(10) Pub. No.: US 2019/0247302 A1
(43) Pub. Date: Aug. 15, 2019

(54) MATERIALS AND METHODS FOR TREATING OPHTHALMIC INFLAMMATION

(71) Applicant: University of Miami, Miami, FL (US)

(72) Inventors: Robert B. Levy, Miami, FL (US); Victor L. Perez, Miami, FL (US); Claes Wahlestedt, Miami, FL (US); Casey O. Lightbourn, Miami, FL (US); Sabrina Copsel, Miami, FL (US); Henry Barreras, Miami, FL (US); Stephanie Duffort, Miami, FL (US)

(73) Assignee: University of Miami, Miami, FL (US)

(21) Appl. No.: 16/343,373

(22) PCT Filed: Oct. 20, 2017
(86) PCT No.: PCT/US2017/057621

§ 371 Date: Apr. 18, 2019

Related U.S. Application Data
(60) Provisional application No. 62/411,183, filed on Oct. 21, 2016.

(51) Int. Cl. A61K 9/00; A61P 27/02; A61P 29/00; A61K 31/724; A61K 31/554; A61K 31/437

(52) U.S. Cl. CPC A61K 9/0048; A61P 27/02; A61K 31/437; A61K 31/724; A61K 31/554; A61P 29/00

(57) ABSTRACT

The disclosure provides an ophthalmic formulation comprising a bromodomain inhibitor, a polar aprotic solvent (e.g., dimethyl sulfoxide (DMSO)), polysorbate, salt, and Captisol, as well as a method of treating or preventing ophthalmic inflammation in a subject in need thereof comprising locally administering the formulation to the eye of the subject.

### CROSS-REFERENCE TO RELATED APPLICATIONS

This application claims the benefit of U.S. Provisional Application No. 62/411,183, filed Oct. 21, 2016, the entire contents of which are incorporated herein by reference.

### STATEMENT OF GOVERNMENT INTEREST

This invention was made with government support under NEI RO1 EY024484 awarded by the National Institutes of Health. The government has certain rights in the invention.

### FIELD OF THE INVENTION

The disclosure is related to formulations comprising bromodomain inhibitors and methods of use.

### BACKGROUND

Ophthalmic inflammation is a common condition that causes discomfort, affects vision, and can lead to significant complications in the eye. Inflammation is associated with a variety of ailments including, but not limited to, conjunctivitis, uveitis, cyclitis, scleritis, episcleritis, neuritis, keratitis, blepharitis, corneal and/or conjunctival ulcer, infection, and also can result from deficient tear production, allergies, abrasions, and surgical operations. Keratoconjunctivitis sicca (KCS), also known as dry eye, is characterized by dryness of the conjunctiva and cornea and is caused by, e.g., dysfunction of the tear-secreting glands or ocular surface.

Keratoconjunctivitis sicca often manifests as itching, redness, the sensation of having a foreign object in the eye, and/or fatigue. It has been estimated that approximately 1 million to 4.3 million people aged 65 to 84 suffer from dry eye. Cornea/External Disease Preferred Practice Pattern Guidelines, Dry Eye Syndrome PPP, American Academy of Ophthalmology, October 2013 (www.aao.org/preferred-practice-pattern/dry-eye-syndrome-ppp--2013). A significant percentage of patients that receive an allogeneic hematopoietic stem transplant (HSCT/bone marrow transplant) also suffer from ocular inflammation that leads to severe KCS and ocular surface damage. If left untreated, keratoconjunctivitis sicca can lead to chronic ocular surface inflammation, damage to the ocular surface, irregular astigmatism, scarring of the conjunctiva and cornea, and improper wound healing.

Current treatments for dry eye include artificial tears or hydrating gels, topical azithromycin, topical cyclosporine (e.g., Restasis™), corticosteroids, anti-integrin therapy (Xiidra™, Lifitegrast 5%), autologous serum tears, punctal occlusion to increase tear residency, and cholinergic agonists. Current treatments have a variety of drawbacks. For example, multiple applications of artificial tears are required per day to obtain relief, punctal plugs often release from the tear duct, and long-term use of topical corticosteroids may be associated with glaucoma, cataracts, and other steroid-related side effects. See, e.g., Mah et al., Clin Ophthalmol. 2012; 6: 1971-1976. In subjects at risk for or suffering from increased intraocular pressure, steroids may pose an unacceptable risk of steroid-associated glaucoma. Additionally, Restasis™ and Xiidra™ predominantly target the recruitment and activation of infiltrating T cells of the ocular surface and, therefore, are limited in their efficacy to treat ocular inflammation. Accordingly, there exists a need for alternate treatment options for ophthalmic inflammation and, in particular, keratoconjunctivitis sicca.

### SUMMARY OF THE INVENTION

The disclosure provides an ophthalmic formulation comprising a bromodomain inhibitor, a polar aprotic solvent (e.g., dimethyl sulfoxide (DMSO)), polysorbate, salt, and Captisol. In various embodiments, the bromodomain inhibitor is JQ1, IBET-151, or EP313; the polysorbate is polysorbate-80; and/or the salt is sodium chloride. The disclosure further provides a method of treating or preventing ophthalmic inflammation in a subject in need thereof. The method comprises locally administering (e.g., topically or subconjunctival) the formulation to an eye of the subject. In one embodiment, the ophthalmic inflammation is keratoconjunctivitis sicca or keratitis.

### DESCRIPTION OF THE FIGURES

FIGS. 1A-1C are bar graphs illustrating inhibition of TNFα production in a culture of RAW cells (macrophage cell line) by bromodomain inhibitors. FIG. 1A illustrates TNFα mRNA levels after 3.5 hours of LPS treatment with and without bromodomain inhibitor (BRDi) treatment. Y axis=TNFα/GAPDH mRNA level (times respect to control); X axis (left to right)=control, EP313 50 nM, EP313 500 nM, LPS, LPS+EP313 50 nM, LPS+EP313 500 nM, control, JQ1 50 nM, JQ1 500 nM, LPS, LPS+JQ1 50 nM, LPS+JQ1 500 nM, control, IBET151 50 nM, IBET151 500 nM, LPS, LPS+IBET151 50 nM, LPS+IBET151 500 nM.

FIG. 1B illustrates TNFα mRNA levels in response to 3.5 h of BRDi treatment alone. Y axis=TNF/GAPDH mRNA level (times respect to control); X axis (left to right)=control, EP313 50 nM, EP313 500 nM, JQ1 50 nM, JQ1 500 nM, IBET151 50 nM, IBET151 500 nM.

FIG. 1C illustrates TNFα mRNA levels after 3.5 hours of LPS treatment with BRDi treatment. Y axis=TNFα/GAPDH mRNA level (times respect to control); X axis (left to right)=control, LPS, LPS+EP313 50 nM, LPS+EP313 500 nM, LPS+JQ1 50 nM, LPS+JQ1 500 nM, LPS+IBET151 50 nM, LPS+IBET151 500 nM.

FIG. 2 is a scatterplot graph illustrating TNFα protein inhibition by JQ1, IBET151, and EP313, demonstrated by intracellular monoclonal antibody staining of TNFα and analyzed by flow cytometry. Y axis=TNFα percentage of P1 gate; X axis=no LPS/no BRDi, 1 μg/ml LPS/no BRDi, BRDI 500 nM LPS, BRDI 250 nM LPS, BRDI 125 nM LPS, BRDI 67.5 Nm LPS. Triangles=JQ1; squares=IBET151; circles=EP313.

FIG. 3 illustrates a study design to characterize the effect of systemically administered BRDi on infiltrate and/or cytokine production by ocular infiltrating cells following corneal LPS induced keratitis.

FIGS. 4A-4B illustrate the results of an in vivo study wherein BRDi were systemically administered to a clinically relevant model of ocular inflammation. FIG. 4A comprises four panels illustrating flow cytometric analysis gated on CD11b corneal infiltrate. Panel 1=no BRDi; Panel 2=IBET151 (10 mg/kg); Panel 3=EP313 (10 mg/kg); Panel 4=EP313 (30 mg/kg). Y-axis=TNFα FITC-A; X axis=FSC-A. FIG. 4B is two bar graphs summarizing flow cytometric protein staining and cell numbers in corneal infiltrate. Panel 1 illustrates TNFα percentage of CD11b (Y-axis) in response to treatment with (left to right on X axis) no BRDi, IBET151 (10 mg/kg), EP313 (10 mg/kg), and EP313 (30 mg/kg). Panel 2 illustrates cell number, TNFα CD11b (Y-axis) in response to treatment with (left to right on X axis) no BRDi, IBET151 (10 mg/kg), EP313 (10 mg/kg), and EP313 (30 mg/kg).

FIG. 5 is a bar graph illustrating the effect of prior formulations on corneal clarity and corneal edema following LPS-induced keratitis. Y axis=clinical haze score; X axis=time after application of LPS+formulations (IBET151, topical (solid bar); IBET151, subconjunctival (hashed bar); solvent, subconjunctival (dotted bar)). The data shows that local administration of IBET151 using prior formulations exacerbates corneal pacification following LPS-induced keratitis.

FIG. 6 is a bar graph illustrating the effect of a formulation of the instant disclosure (administered via subconjunctival injection) on corneal clarity and corneal edema following LPS-induced keratitis. Y axis=clinical haze score; X axis=time after application of LPS+IBET151 formulation (solid bar); solvent alone (hashed bar); saline (dotted bar)). The data shows that local subconjunctival administration of IBET151 using RLVP.001 prevents corneal opacification following LPS-induced keratitis.

FIG. 7 is a bar graph illustrating the effect of a formulation of the instant disclosure (topical administration) on corneal clarity and corneal edema following LPS-induced keratitis. Y axis=clinical haze score; X axis=time after application of LPS+JQ1 formulation (topical, hashed bar); solvent alone (subconjunctival, dotted bar)). The data shows that topical administration of JQ1 using RLVP.001 prevents corneal opacification following LPS-induced keratitis.

FIGS. 8A-8C are a bar graph illustrating the effect of BRDi on a human ocular surface primary parenchymal cell line. FIG. 8A illustrates cytokine/GAPDH mRNA levels (ratio to no treatment) (Y-axis) of IL-8, IL-1β, IL-6, TNFα, and CCL2 in HCEC administered no treatment (solid bars), three hours after exposure to LPS (gray bar), and three hours after exposure to LPS and JQ1 (500 nM, hashed bar). FIG. 8B illustrates cytokine/GAPDH mRNA levels (ratio to no treatment) (Y-axis) of IL-8, IL-1β, and IL-6 in keratocytes administered no treatment (solid bars), four hours after exposure to LPS (gray bar), four hours after exposure to LPS and JQ1 (500 nM, hashed bar), and four hours after exposure to LPS and EP313 (500 nM, checked bar). FIG. 8C illustrates cytokine/GAPDH mRNA levels (ratio to no treatment) (Y-axis) of CCL2 in keratocytes administered no treatment (solid bars), four hours after exposure to LPS (gray bar), four hours after exposure to LPS and JQ1 (500 nM, hashed bar), and four hours after exposure to LPS and EP313 (500 nM, checked bar). The data shows epigenetic regulation of human ocular surface primary parenchymal cell line responses to LPS-induced inflammation.

FIGS. 9A-9B depicts chemical structures of JQ1 (FIG. 9A) and IBET151 (FIG. 9B).

FIG. 10 shows a table depicting BETi EP313 administration schedules for three independent experiments analyzing ocular GVHD (OGVHD). Experiment OGVHD #28 includes data from a therapeutic analysis of BETi for treatment of OGVHD in a MUD transplant involving B6--->C3H.SW mice in which BETi was administered topically 2×/day and 3×/week (M/W/F). Experiment OGVHD #33 includes data from a therapeutic analysis of BETi for treatment of OGVHD in the MUD HSCT mouse model (B6--->C3H.SW) in which BETi which was administered topically 2×/day (M T W Th F) and subconjunctival every other Friday. The third experiment OGVHD #36 includes data from a therapeutic analysis of BETi for treatment of OGVHD in the MUD HSCT model (B6--->C3HSW) in which BETi which was administered 1×/day on W and Th and subconjunctival on Mondays and Fridays.

FIGS. 11A-11B show a modified dot plot and graph, respectively, representing data of a therapeutic analysis of BETi for treatment of ocular GVHD from Experiment OGVHD #28 in B6--->C3H. SW mice. Open circles represent BM^{CD45.1} only; open squares represent BM^{CD45.1}+T cells^{B6Thy1.1}; open triangles represent B3+EP313; and black inverted triangles represent B2 T cells+Solvent. FIG. 11A is a modified dot plot showing clinical lid score data recorded at Day 51, in which each data point represents an individual eye. FIG. 11B is a graph showing clinical lid score data for each of the experimental groups depicted in FIG. 11A taken over the duration of the experiment.

FIG. 12 is a graph showing data from experiment OGVHD #33 in a MUD HSCT mouse model (B6--->C3H. SW). Open circles represent BM+Solvent; open squares represent BM+EP313; open triangles represent BM+T cells+Solvent; and black inverted triangles represent BM+T cells+EP313. These data validate that there is an effect of the BETi being assessed, and suggest that modification of the treatment regimen may be able to increase efficacy.

FIGS. 13A-13C show graphs representing data of a therapeutic analysis of BETi for treatment of ocular GVHD from experiment OGVHD #36 in a MUD HSCT Model (B6--->C3HSW). FIG. 13A is a graph showing percentage initial starting weight versus time from Day 0 to Day 56. FIG. 13B is a graph showing systemic clinical score versus time from Day 0 to Day 56. FIG. 13C is a graph showing percent survival versus time from Day 0 to Day 56. Open circles represent A1 BM; open squares represent B1 BM+T cells Solvent; black triangles represent B2 BM+T cells EP313. These data sets illustrate the progress of systemic GVHD in this mouse model, and demonstrate that the local administration of EP313 does not affect the development of this GVHD.

FIGS. 14A-14B are a graph and modified dot plot, respectively, showing data from experiment OGVHD #36 in a MUD HSCT mouse model (B6J--->C3HSW). FIG. 14A is a graph showing clinical lid score versus time from Day 0 to Day 63. FIG. 14B is a modified dot plot showing clinical lid score versus time from Day 0 to Day 63 where each point represents individual lid scores from each of the three groups of transplanted animals over eight weeks post-hematopoietic stem cell transplant. It is important to note that as the experiment progressed, more animals died. As a result, the lid scores for these deceased animals do not appear at later times and as indicated, by Day 56, there were 5 mice remaining alive in the EP313 treated group, and 4 mice remaining alive in the solvent treated group.

### DETAILED DESCRIPTION OF THE INVENTION

Provided herein are improved formulations and methods of use for treating ophthalmologic inflammation, including, e.g., dry eye disease (keratoconjunctivitis sicca), ocular surface inflammation, inflammation resulting from allergies, keratitis, conjunctivitis, uveitis, inflammation associated with diabetic retinopathy, inflammation associated with age related macular degeneration, cyclitis, scleritis, episcleritis, blepharitis, inflammation associated with corneal and/or conjunctival ulcer, inflammation associated with abrasion or wound to the eye, inflammation associated with ophthalmic graft versus host disease (GVHD), or inflammation associated with ophthalmologic surgery. The development of new treatment options for ophthalmologic inflammation is complicated by unique requirements for direct administration to the eye. Formulations, for instance, must comprise a sufficient concentration of therapeutic to provide a beneficial effect in an extremely small volume of solution, disperse along the ocular surface to maximize delivery in topical applications, and absorb into adjacent tissues, as well as minimize impact on vision and intraocular pressure. Anti-inflammatories and formulation components that may be suitable for other areas of the body may not be suitable for use in the eye.

The disclosure relates to the application of epigenetic regulators, specifically bromodomain inhibitors, to treat ophthalmologic inflammation. The disclosure also relates to ophthalmic formulations comprising bromodomain inhibitors for local delivery in the eye.

Bromodomains are amino acid domains that recognize acetylated lysine residues. See, e.g., Ntranos et al., Neuroscience Letters, 625, 4-10, 2016. Bromodomain inhibitors target the binding pocket of bromodomain-containing molecules, preventing the ability of the bromodomain-containing molecules to promote gene transcription. An example of a bromodomain family is the Bromodomain and Extraterminal Domain family (BET). Examples of bromodomain-containing proteins include, but are not limited to, ASH1L, ATAD2A/B, BAZ1A/B, BAZ2A/B, BRD1, BRD2, BRD3, BRD4, BRDT, BRD7, BRD8A/B, BRD9, BRPF1A/B, BRPF3A, BRWD3, CECR2, CREBBP, EP300, FALZ, GCN5L2, MLL, PB1, PCAF, PHIP, PRKCBP1, SMARCA2A/B, SMARCA4, SP 100/SP 110/SP 140, TAF 1/TAAFiL, TRIM24/28/33/66, WDR9, and ZMYND11. Muller et al., Expert Rev Mol Med., 13, e29, 2011. Examples of bromodomain inhibitors include, but are not limited to, JQ1, IBET (IBET151), and EP313. Additional examples include, e.g., GSK525762, OTX015 (CAS 202590-98-5), acetyl lysine mimetics, GW841819X, and ischemin. Bromodomain inhibitors are also described in Ciceri et al, Nature Chemical Biology 10, 305-312, 2014.

In various aspects, the disclosure provides an ophthalmic formulation comprising a bromodomain inhibitor (e.g., JQ1, IBET151, or EP313), a polar aprotic solvent (e.g., dimethyl sulfoxide (DMSO)), polysorbate, salt, and Captisol®. Optionally the polysorbate is polysorbate-80 (i.e., TWEEN-80). In various aspects, the salt is mono-, di- or trisodium phosphate, sodium chloride, potassium chloride, or combinations thereof. Optionally, the salt is sodium chloride.

Captisol® is a mixture of polyanionic-cyclodextrin derivatives of a sodium sulfonate salt tethered to the lipophilic cavity by a butyl ether group, or sulfobutyl ether (SBE). The sulfobutyl ether (SBE) substituent is introduced at the 2, 3, and 6 positions in one or more of the glucopyranose units in the cyclodextrin structure. Captisol® is otherwise described as sulfobutylether-β-cyclodextrin having the molecular formula C₄₂H₇₀-nO₃₅ (C₄H₈S₃Na)n; where n=the average degree of substitution; CAS Number 182410-00-0. The structure of CAPTISOL® is provided below, wherein R═(H)₂₁-n or (CH2 CH2 CH2 CH2 SO₂ONa)n, where n=6.2 to 6.9.

See CAPTISOL® product brochure available at www.zerista.s3.amazonaws.com/item_files/3f69/attachments/25 850/original/brochure-a7075472-059c-4470-bd96-52e53e263de3.pdf.

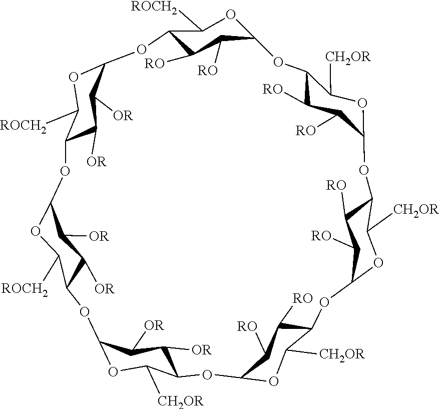

In various embodiments, the formulation comprises 0.1%-1% polar aprotic solvent (e.g., DMSO), 0.1%-1% polysorbate (e.g., polysorbate-80), 0.1%-1.5% salt (e.g., sodium chloride), and 1%-2.5% Captisol®. For example, in one aspect, the formulation comprises 0.5% DMSO, 0.5% polysorbate-80, 0.9% sodium chloride, and 1.8% Captisol®. Optionally, the formulation comprises 0.5%-5% bromodomain inhibitor (e.g., JQ1, IBET151, or EP313).

Polysorbate serves as a solubilizing agent, emulsifier, and/or a surfactant in formulations, and alternate or additional solubilizing agents, emulsifiers, and/or surfactants are contemplated for use in the formulation, such as, e.g., tyloxapol or poloxamer. In addition to the components set forth above, the ophthalmic formulations of the present disclosure may contain additional components, such as buffering agents (e.g., phosphate buffers, borate buffers, citrate buffers, tartrate buffers, acetate buffers, amino acids, sodium acetate, sodium citrate), tonicity agents (e.g., saccharides (such as sorbitol, glucose, or mannitol), polyethylene glycol, or propylene glycol), preservatives, antiseptics, analgesics, pH modifiers (e.g., acetic acid, phosphoric acid, sodium hydroxide, or potassium hydroxide), chelating agents (e.g., sodium edetate or sodium citrate), and the like.

Also provided is a method of treating or preventing ophthalmic inflammation in a subject (e.g., human) in need thereof. The method comprises locally administering the formulation described herein to the eye of the subject. The ophthalmic inflammation is optionally keratoconjunctivitis sicca, ocular surface inflammation, inflammation resulting from allergies, keratitis, conjunctivitis, uveitis, inflammation associated with diabetic retinopathy, inflammation associated with age related macular degeneration, inflammation associated with nondiabetic macular edema, cyclitis, scleritis, choroiditis, episcleritis, blepharitis, inflammation associated with corneal and/or conjunctival ulcer, inflammation associated with infection (e.g., bacterial or viral infection), inflammation associated with abrasion or wound to the eye, inflammation associated with ophthalmic GVHD, or inflammation associated with ophthalmologic surgery (e.g., cataract surgery, retinal surgery, refractive surgery, or corneal surgery). In a preferred embodiment, the ophthalmic inflammation is keratoconjunctivitis sicca, keratitis, or inflammation associated with ophthalmic GVHD.

The formulation is locally administered to the eye. Local administration refers to direct application to the eye, not systemic delivery. Systemic delivery is contemplated in various aspects, which are distinct from local administration. In various embodiments, the formulation is administered topically, subconjunctivally, retrobulbarly, periocularly, subretinally, suprachoroidally or intraocularly. The formulation is administered in an amount effective to achieve a beneficial response in a clinically reasonable amount of time. For example, the formulation is administered in an amount effective to ameliorate ocular inflammation or symptoms thereof, in whole or in part, and/or protect, in whole or in part, against ocular inflammation or symptoms thereof (e.g., protect against increased severity of the inflammation). Inflammation is diagnosed and monitored using a variety of techniques, including general eye exams, slit lamp examination, dilated fundus examination, corneal topography, lipid layer analysis, objective red eye scaling, corneal haze examination, staining, and the like. Generally, the formulation will be administered as soon as inflammation or infection is detected or ocular surgery is completed. The formulation also may be administered before inflammation or infection is detected or before or during ocular surgery.

Dosage will depend upon a variety of factors, including the strength of the particular bromodomain inhibitor employed, the condition or disease state to be treated, and the amount and location of inflammation. The size of the dose also will be determined by the route, timing and frequency of administration as well as the existence, nature, and extent of any adverse side effects that might accompany the administration of a particular bromodomain inhibitor and the desired physiological effect. For topical application, the formulation may be packaged in eye drop bottles and administered as drops. A single administration (i.e., a single dose) of the formulation may include a single drop, two drops, three drops or more into the eyes of the subject. Generally, the dose of bromodomain inhibitor is about 0.5% to about 5% provided in drop form, although intraocular injection also is contemplated.

Various conditions or disease states, in particular, chronic conditions or disease states, may require prolonged treatment involving multiple administrations. For example, in some embodiments, the ophthalmic formulation is administered at predetermined time intervals over an extended period of time. In various aspects, ophthalmic formulation is administered once a day, twice a day, three times per day, four times per day, or more. Optionally, the ophthalmic formulation is administered every other day. In some embodiments, the ophthalmic formulation is administered over a treatment period of one week, two weeks, three weeks, one month, two months, three months, six months, nine months, twelve months, 18 months, or more. In severe or chronic cases, the ophthalmic formulation is administered for a more extended period of time, including throughout the duration of the subject's life. Optionally, the formulation is administered for a period of time then temporarily suspended before further treatment (i.e., a dosing holiday). Also optionally, an initial therapeutic dose may be administered to the subject for a treatment period, after which a maintenance dose (typically lower than the therapeutic dose) is administered for a maintenance period of time. In this regard, the dosage and/or the frequency of administration is optionally reduced to a regimen that maintains the inflammation or symptoms thereof at an acceptable level.

In various aspects, the formulation is provided in a sustained-release delivery system or device. Implants and devices are disclosed in, e.g., U.S. Pat. Nos. 5,443,505, 4,853,224, 4,997,652, 5,554,187, 4,863,457, 5,098,443 and 5,725,493. An implantable device, e.g., a mechanical reservoir, an intraocular device or an extraocular device with an intraocular conduit (e.g., 100 μm-1 mm in diameter) can be used. Matrix-type delivery systems also are suitable for delivering the formulation. See e.g., Ueno et al., “Ocular Pharmacology of Drug Release Devices”, in Controlled Drug Delivery, Bruck, ed., vol. II, Chap 4, CRC Press Inc. (1983) (incorporated herein by reference in its entirety). Examples of matrix-type delivery systems include soft contact lenses impregnated or soaked with the bromodomain inhibitor composition, as well as biodegradable implants comprising, e.g., poly(vinyl alcohol), polymers and copolymers of polyacrylamide, ethylacrylate, vinylpyrrolidone, polypeptides, polysaccharides, and/or crosslinked hyaluronic acid.

The method optionally further comprises co-administration of other pharmaceutically active agents. By “co-administration” is meant administration before, concurrently with, e.g., in combination with the bromodomain inhibitor in the same formulation or in separate formulations, or after administration of a bromodomain inhibitor as described above. For example, corticosteroids, prednisone, dexamethasone, or triamcinalone acetinide, or noncorticosteroid anti-inflammatory compounds, such as ibuprofen, can be co-administered. In addition, a steroid sparing agent, such as Restasis™ (cyclosporine ophthalmic emulsion, 0.05%) and Lifitegrast, can be co-administered. Similarly, vitamins and minerals, anti-oxidants (e.g., carotenoids), and micronutrients can be co-administered. Additionally, the bromodomain inhibitors may be co-administered with agents that selectively promote immune down regulation, such as regulatory T cell expansion.

### Examples

While not wishing to be bound by any particular theory, bromodomain inhibitors interfere with the functional activities of both infiltrating inflammatory as well as ocular parenchymal cells, such as cornea/conjunctiva epithelial cells, keratocytes and conjunctival stromal elements. Bromodomain inhibitors target the binding pocket of bromodomain-containing molecules, preventing bromodomain-containing molecules to promote transcription of, e.g., inflammatory molecules IL-1β, TNFα, IL-6, IL-17, INFγ, CCL2, CXCL10, IL-4 and IL-10, associated with ophthalmologic inflammation, in particular keratoconjunctivitis sicca. The inhibitors also target the NFKβ pathway associated with induction of inflammatory proteins.

A novel pre-clinical MHC-matched allogeneic hematopoietic stem cell transplantation HSCT model (“MUD”—Matched Unrelated Donor) was developed that results in systemic and ocular GVHD with onset kinetics similar to that observed in patients. C3H.SW (H-2b, Ly9.1+) mice were transplanted with B6 (H-2b, Ly9.1-) T-cell depleted bone marrow (TCD-BM) and T cells. First, only mice that received TCD-BM+T cells underwent weight loss and began exhibiting clinical signs of GVHD ˜3 wks post-HSCT. Second, the mice also developed ocular surface disease evidenced by progression of ocular surface damage characterized by increased lid margin swelling, conjunctiva inflammation and lacrimal gland fibrosis that is associated with poor tear production, corneal staining and ulceration by week 6 after transplantation. Ocular GVHD occurs in >60% of patients that undergo allogeneic HSCT and similar to our pre-clinical model, is also characterized by dry eye, meibomian gland dysfunction, conjunctiva damage, punctate keratopathy, corneal ulceration and perforation.

Histological analyses of the ocular adnexa of these animals demonstrated that only mice that developed systemic and ocular GVHD exhibited corneal thickening and epithelial irregularity, as well as dense inflammatory cell infiltrates. Significantly, in contrast to the CD8>CD4 systemic GVHD immune phenotype, the CD4>CD8 ratio immune phenotype in the ocular compartment is distinct. Furthermore, the detection of IFN-γ and TNF-α, suggests that Th1 allo-rx effector cells and M1 inflammatory mΦ are involved in ocular GVHD. Thymic injury seen in these mice suggests that the failure of intrathymic central tolerance may result in the involvement of donor and/or host pathogenic T cells with self-rx to ocular antigens. Based on these results, it was believed that both allo-rx donor T cells and/or self-rx T cells infiltrate the ocular surface and orchestrate the recruitment of inflammatory M1 mΦ that contribute to ocular damage.

Intra-vital time-lapsed fluorescence microscopy was used to non-invasively monitor the tempo of EGFP+ transplanted donor T cells in the eye. C3H.SW mice were transplanted using B6-EGFP (H2b) donors and EGFP expression was correlated with systemic and ocular GVHD onset and progression. The data demonstrated that recruitment of EGFP+cells into the ocular adnexa begins between 3-4 weeks after transplantation, and inflammation of the lid margin appears to be a very sensitive marker of onset of disease. To address the requirement for CD4 and CD8 in the induction of ocular GVHD, transplants were performed using purified CD4 and CD8 subsets obtained from B6 donor mice. In contrast to control mice that received TCD-BM, mice that received both T cells developed systemic and ocular GVHD as previously described. However, the development of ocular GVHD was clinically more significant in mice that received CD4 T cells only in comparison to CD8 T cells transplanted mice, suggesting that CD4 T cell subset may be more relevant in the development of ocular GVHD.

Furthermore, chemokine and cytokine RNA findings from ocular tissue in mice with GVHD found, a) T cell chemo-attractant, CXCL10 and CXCR3 expression b) cytokines reflective of M1 inflammatory mΦ and Th1 effector cells. The presence of these effector molecules has been demonstrated at the ocular surface of patients with ocular GVHD. To test the hypothesis that a potential mechanism of inflammatory T cell recruitment into the ocular adnexa of mice with GVHD may be dependent on the CXCL10/CXCR3 axis, transplants using T cells from B6-CXCR3 knock out mice were performed. The data demonstrated that mice transplanted with CXCR3 knock out T cells developed less ocular GVHD in contrast to mice that received wild type T cells. These data suggest that inhibiting the recruitment of ocular GVHD T cells using anti-chemokine receptors compounds may be a strategic approach to prevent or treat ocular GVHD, especially if it can be delivered locally.

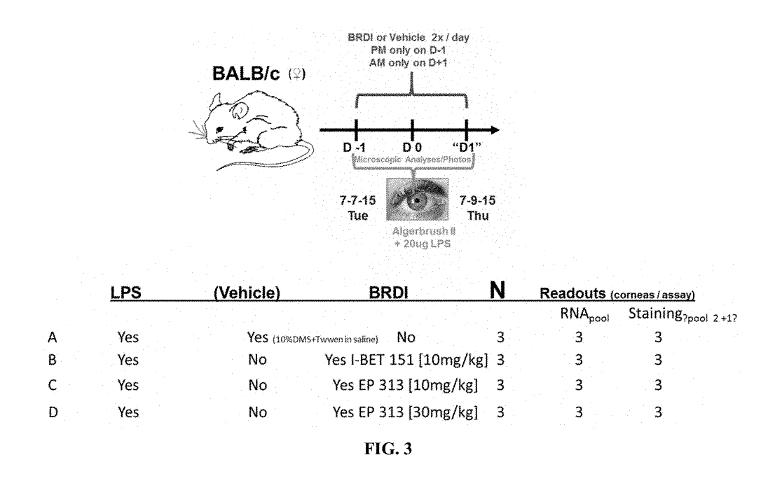

Studies with epigenetic regulators were conducted to investigate if such compounds altered the phenotype and/or function of inflammatory cells recruited to this compartment. To test the role of bromodomain inhibition in ocular inflammation, an in vivo model of corneal inflammation induced by LPS was developed. BRDi compounds were administered systemically by intraperitoneal injection beginning ˜15 hours prior to the induction of LPS keratitis (FIG. 3).

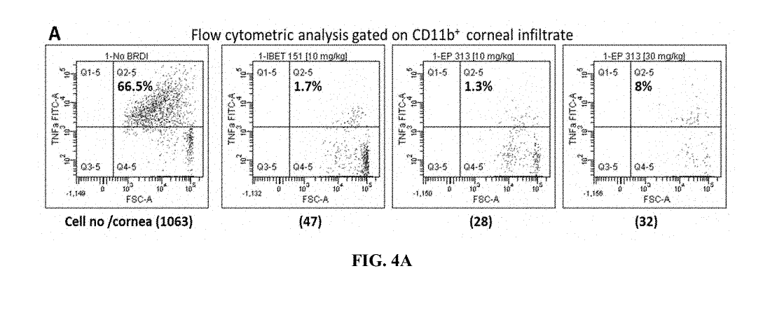

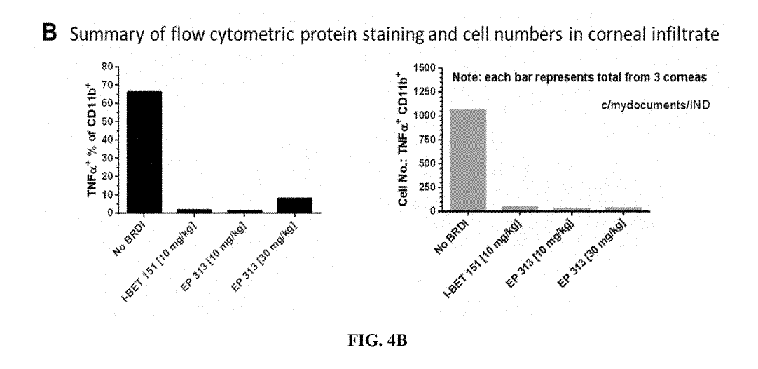

This model represents clinically relevant ophthalmic pathological damage caused by bacterial infections. Corneal infiltrates were collected from multiple corneas and analyzed by flow cytometry for the intracellular production of TNFα in CD11b+(macrophage) ocular infiltrating cells. The data demonstrated that, similar to the results of the in vitro experiments described herein, bromodomain inhibitors effectively reduce the production of pro-inflammatory cytokines in vivo, specifically in the eye (FIG. 4A-4B).

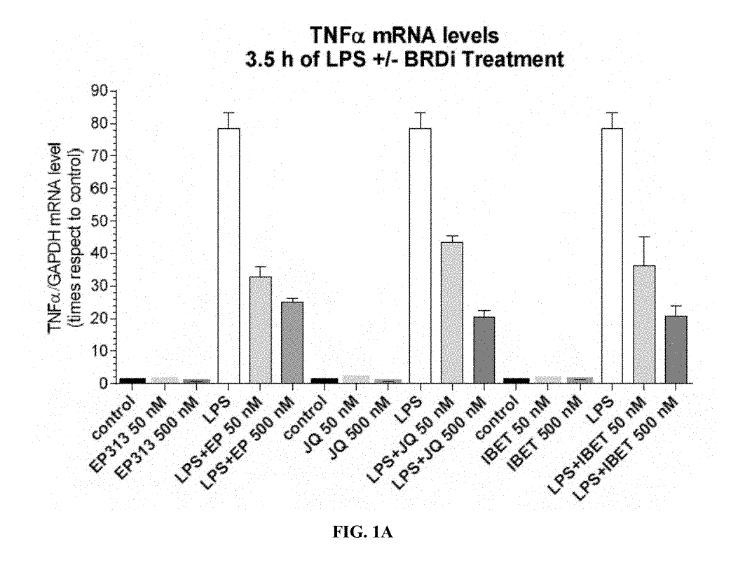

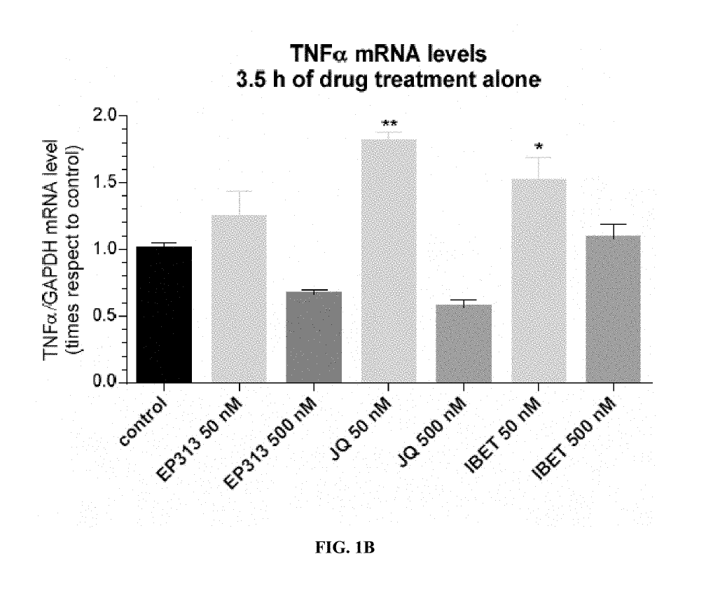

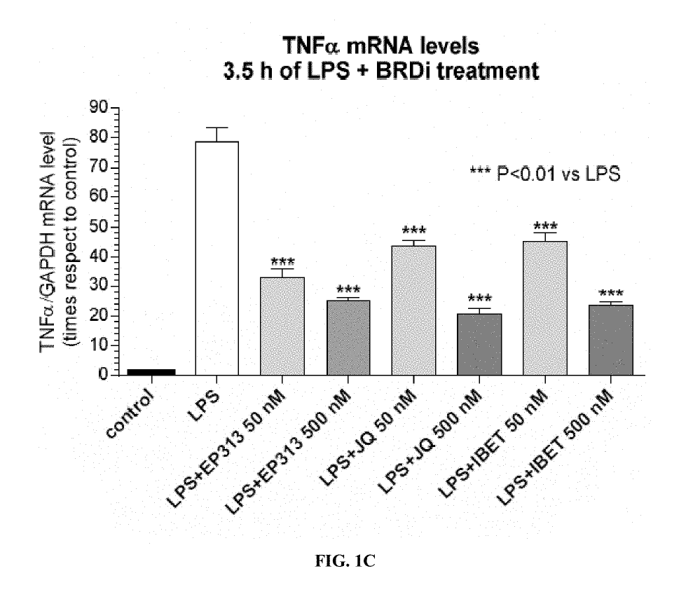

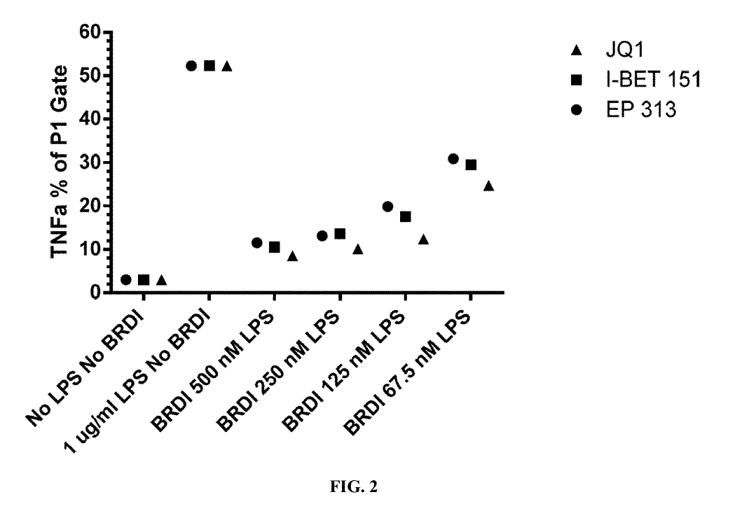

The ability of EP313 to regulate several cytokines was initially examined using a macrophage cell line (RAW cells) after stimulating these cells with LPS to induce inflammatory molecules and first demonstrated significant down regulation of TNFα RNA. In vitro data (FIG. 1A-1C) demonstrated that, following stimulation with lipopolysaccharide (LPS—present on the cell walls of bacteria) to induce inflammatory molecules, significant downregulation of TNFα RNA was observed. The production of inflammatory cytokines TNFα, IL-6 and 11-12 by LPS-stimulated primary CD11b+(predominantly macrophages and dendritic cells) spleen cells was downregulated by bromodomain inhibitors (data not shown). Studies then examined protein production and confirmed that TNFα was reduced by treating RAW cells in vitro with multiple bromodomain inhibitor compounds (FIG. 2). The production of TNFα, IL-6 and IL-12 by LPS stimulated primary CD11b+ spleen cells (predominantly macrophages and dendritic cells) was markedly down regulated by EP313 as demonstrated by intracellular cytokine staining. Similar results were detected with RAW cells using additional bromodomain inhibitors including JQ1.

To assess the capacity of bromodomain inhibitors to inhibit ocular inflammation, an in vivo model of corneal inflammation induced by LPS was utilized that represents clinically relevant ophthalmic inflammatory response. Bromodomain compounds were first administered systemically by intraperitoneal injection beginning ˜15 hours prior to the induction of LPS keratitis. Infiltrates were collected from multiple corneas and analyzed by flow cytometry for the intracellular production of TNFα in CD1 b+(macrophage) ocular infiltrating cells. Our data demonstrated that similar to the in vitro experiments, bromodomain inhibitors reduced the production of a pro-inflammatory cytokines in vivo, specifically in the ocular compartment.

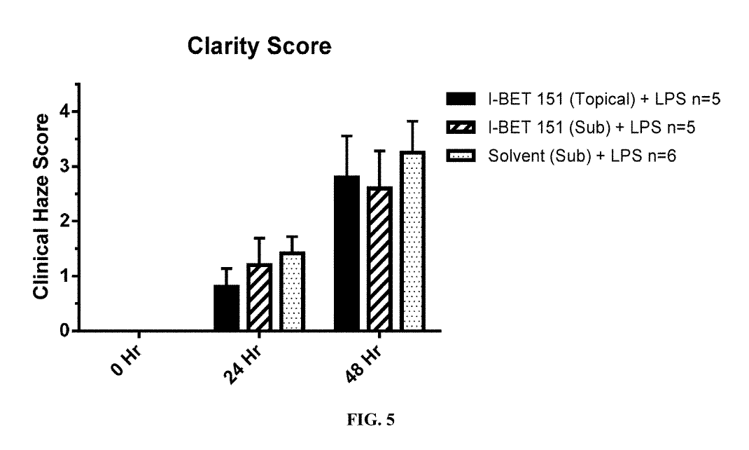

Next, to address whether local administration of bromodomain inhibitors could be used to regulate ocular inflammatory responses, an ophthalmic formulation BRDi compounds was developed. De novo development was required because the solution required to dissolve these non-polar compounds was not amenable to application to the ocular surface, as they require dilution in high concentrations of DMSO and Tween 80. Bromodomain inhibitors applied to the eye with previous formulations (with higher concentrations of DMSO and polysorbate) failed to ameliorate LPS induced keratitis. It was difficult to obtain high concentrations of bromodomain inhibitor in the formulations and, notably, the solvent caused ocular damage illustrated by decrease corneal clarity and increased corneal edema (FIG. 5). As illustrated in FIG. 5, previous formulations with LPS increased the clinical haze score over the course of 48 hours.

To address this issue, a new ophthalmic formulation was developed. The bromodomain inhibitors were successfully diluted at higher concentrations when formulated with DMSO, polysorbate (Tween-80), preservative free 0.9% NaCl, and captisol (Captisol®), which is a polyanionic beta-cyclodextrin derivative providing. The new solution enabled formulation of significantly higher concentrations of the bromodomain inhibitors (JQ1, IBET-151 and EP 313) for local ophthalmic application. Using this new formulation, the concentrations of both DMSO and Tween-80 were able to be reduced, from 5% to 0.5% of each (which is tolerable to the eye). The final solution containing the bromodomain inhibitors was 0.5% DMSO, 0.5% Tween-80 and 1.8% Captisol.

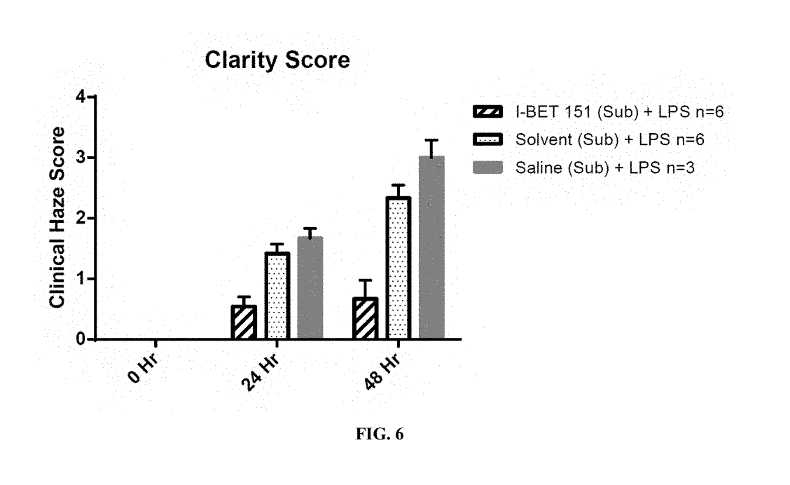

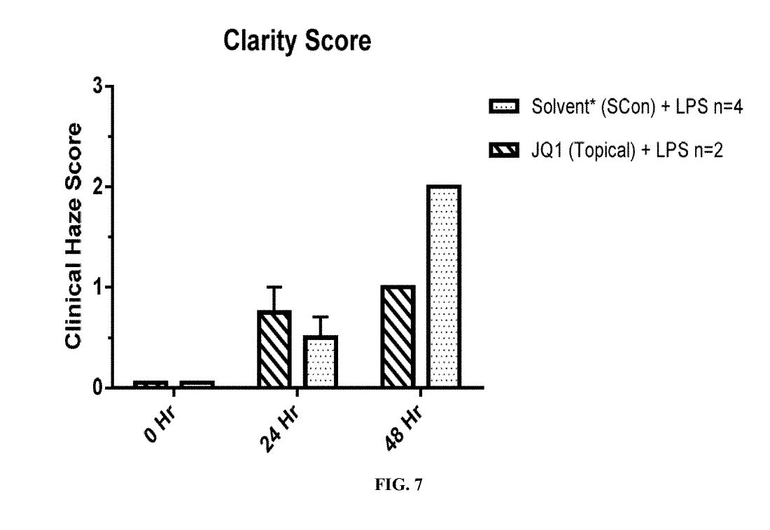

The new formulation comprising bromodomain inhibitor IBET-151 was delivered locally in the eye by subconjunctival injection (20 μg/kg/body weight) just prior and during the induction of in vivo LPS-induced keratitis. The data demonstrated that the local ocular application of IBET-151 ameliorates the development of corneal inflammation, as illustrated by clinically increased clarity (FIG. 6). To confirm that a second bromodomain inhibitor could successfully be used in the ocular compartment to reduce inflammation, JQ1 dissolved in the new ophthalmologic formulation was locally administered (1 μg/μl). The formulation was applied topically, i.e. on the corneal surface 1 drop/3× per day. As observed with the subconjunctival injection with IBET-151, this topical application of the formulation comprising JQ1 also successfully ameliorated the inflammatory response in the cornea of LPS treated animals (FIG. 7).

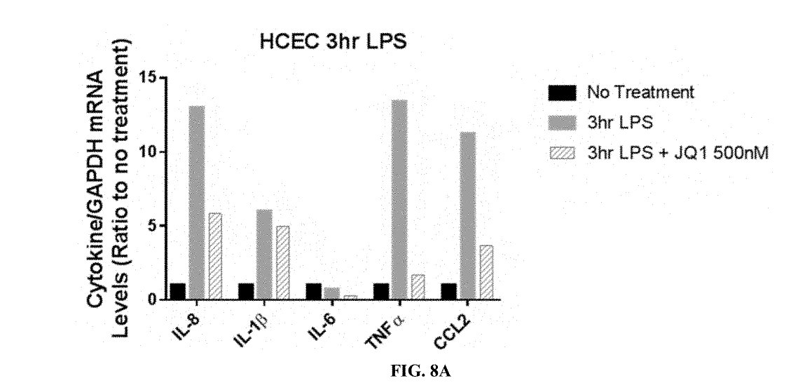

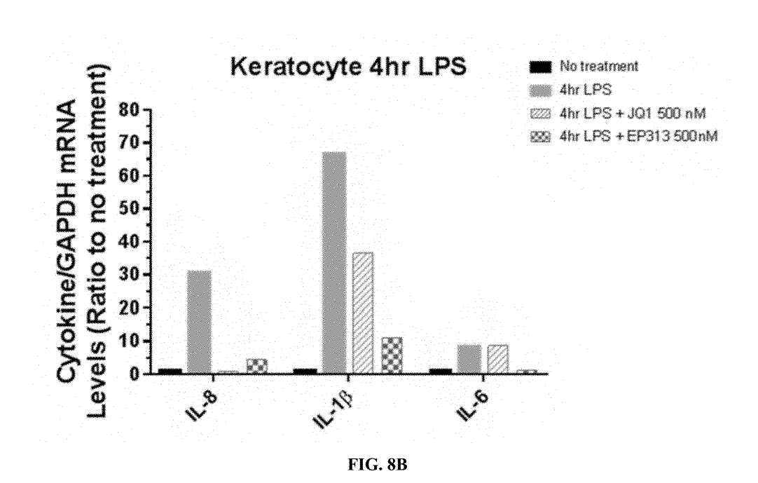

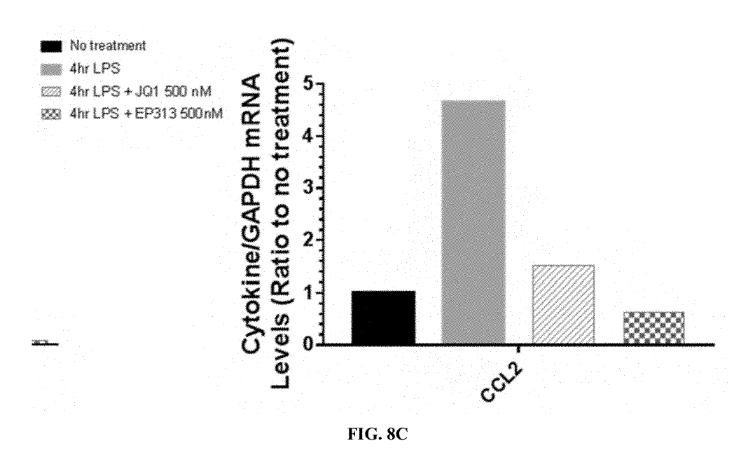

The ability of BRDi compounds to regulate production of dry eye-associated cytokines and chemokines produced by human ocular parenchymal cell populations also was examined. In vitro cells from the cornea and conjunctiva compartments of donated human eyes (Florida Lions Bank) were expanded. Primary cell lines were developed comprising either corneal keratocytes or conjunctival epithelial cells (HCEC) (FIG. 8A-8C). Following 3 hr. exposure to LPS, HCEC produced significant levels of IL-8, IL-13, TNFα, CCL2, and detectable levels of IL-6 RNA (FIG. 8A). HCEC incubated for the 3 hours with JQ1 demonstrated marked decreases in RNA levels of IL-8, TNFα, CCL2 and IL-6, and IL-1β was marginally reduced. Corneal keratocyte cultures were also examined for RNA production and were also regulated by BRDi compounds JQ1 and EP313 (FIGS. 8B and 8C). The levels of IL-13, IL-6, and the chemokine CCL2 were more effectively suppressed by EP313 compared to JQ1. These observations extend the potential of BRDi to inhibit ocular inflammation. Additionally, differences may exist between the families of BRDi compounds such that particular BRDi may be more useful under a specific set of disease conditions.

FIG. 5 is a bar graph illustrating the effect of prior formulations on corneal clarity and corneal edema following LPS-induced keratitis. Y axis=clinical haze score; X axis=time after application of LPS+formulations (IBET151, topical (solid bar); IBET151, subconjunctival (hashed bar); solvent, subconjunctival (dotted bar)). The data shows that local administration of IBET151 using prior formulations demonstrate no significant effect on corneal pacification following LPS-induced keratitis.

FIG. 6 is a bar graph illustrating the effect of a formulation of the instant disclosure (administered via subconjunctival injection) on corneal clarity and corneal edema following LPS-induced keratitis. Y axis=clinical haze score; X axis=time after application of LPS+IBET151 formulation (solid bar); solvent alone (hashed bar); saline (dotted bar)). The data shows that local subconjunctival administration of IBET151 using RLVP.001 prevents corneal opacification following LPS-induced keratitis.

FIG. 7 is a bar graph illustrating the effect of a formulation of the instant disclosure (topical administration) on corneal clarity and corneal edema following LPS-induced keratitis. Y axis=clinical haze score; X axis=time after application of LPS+JQ1 formulation (topical, hashed bar); solvent alone (subconjunctival, dotted bar)). The data shows that topical administration of JQ1 using RLVP.001 prevents corneal opacification following LPS-induced keratitis.

FIGS. 8A-8C are a bar graph illustrating the effect of BRDi on a human ocular surface primary parenchymal cell line. FIG. 8A illustrates cytokine/GAPDH mRNA levels (ratio to no treatment) (Y-axis) of IL-8, IL-1β, IL-6, TNFα, and CCL2 in HCEC administered no treatment (solid bars), three hours after exposure to LPS (gray bar), and three hours after exposure to LPS and JQ1 (500 nM, hashed bar). FIG. 8B illustrates cytokine/GAPDH mRNA levels (ratio to no treatment) (Y-axis) of IL-8, IL-1β, and IL-6 in keratocytes administered no treatment (solid bars), four hours after exposure to LPS (gray bar), four hours after exposure to LPS and JQ1 (500 nM, hashed bar), and four hours after exposure to LPS and EP313 (500 nM, checked bar). FIG. 8C illustrates cytokine/GAPDH mRNA levels (ratio to no treatment) (Y-axis) of CCL2 in keratocytes administered no treatment (solid bars), four hours after exposure to LPS (gray bar), four hours after exposure to LPS and JQ1 (500 nM, hashed bar), and four hours after exposure to LPS and EP313 (500 nM, checked bar). The data shows epigenetic regulation of human ocular surface primary parenchymal cell line responses to LPS-induced inflammation.

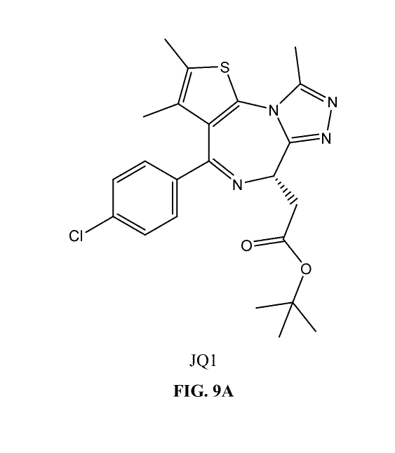

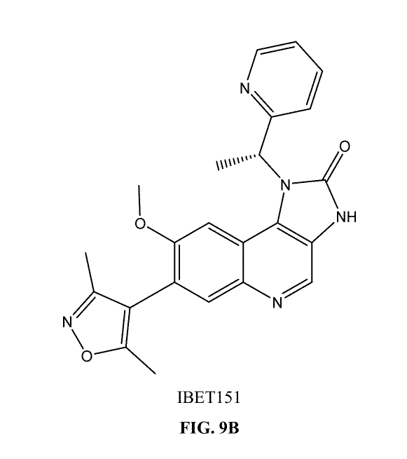

FIGS. 9A-9B depicts chemical structures of JQ1 (FIG. 9A) and IBET151 (FIG. 9B).

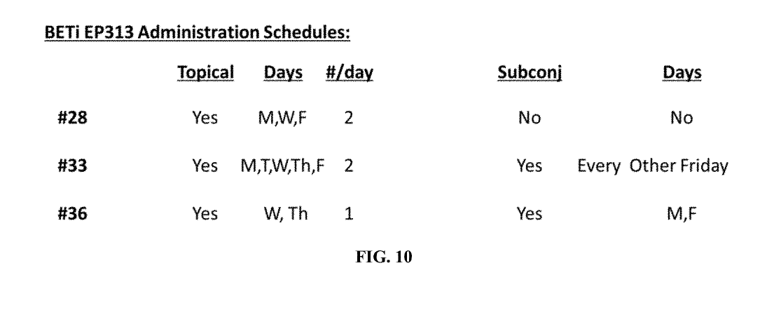

The new formulation comprising bromodomain inhibitor EP313 was delivered locally in the eye by subconjunctival injection in a series of experiments to conduct a therapeutic analysis of EP313 for the treatment of ocular GVHD. FIG. 10 shows a table depicting BETi EP313 administration schedules for three independent experiments analyzing ocular GVHD (OGVHD). Experiment OGVHD #28 includes data from a therapeutic analysis of BETi for treatment of ocular GVHD in MUD B6--->C3H.SW mice in which BETi was administered topically 2×/day and 3×/week (M/W/F). Experiment OGVHD #33 includes data from a therapeutic analysis of BETi which was administered topically 2×/day (M T W Th F) and subconjunctival every other Friday. The third experiment OGVHD #36 includes data from a therapeutic analysis of BETi for treatment which was administered topically 1×/day on W and F and subconjunctival on Mondays and Fridays.

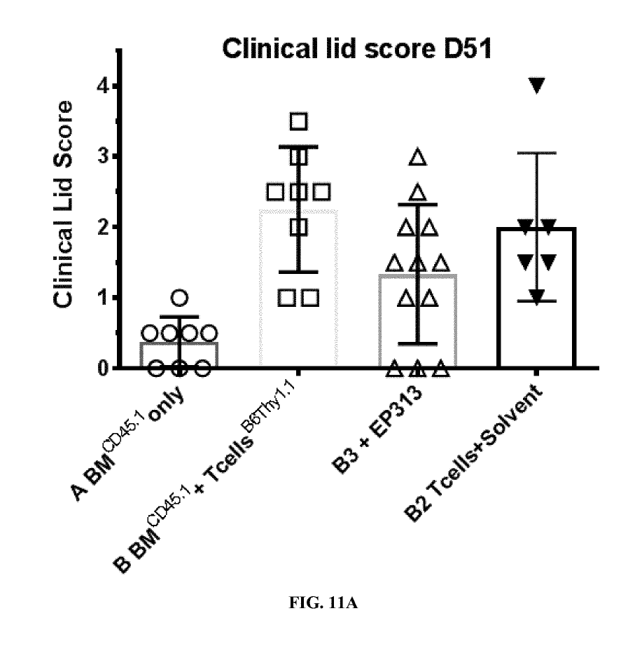

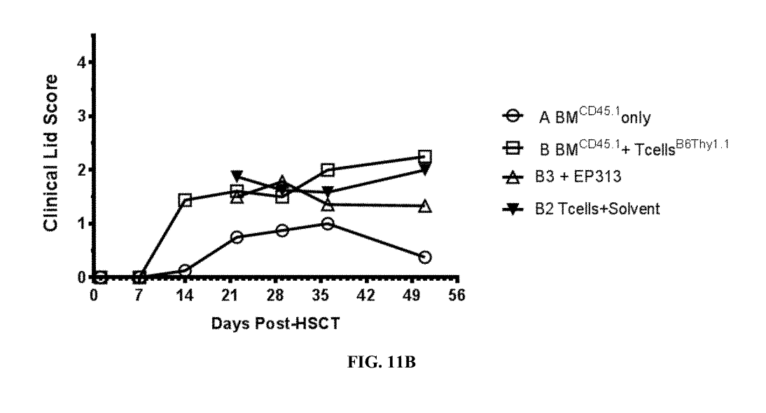

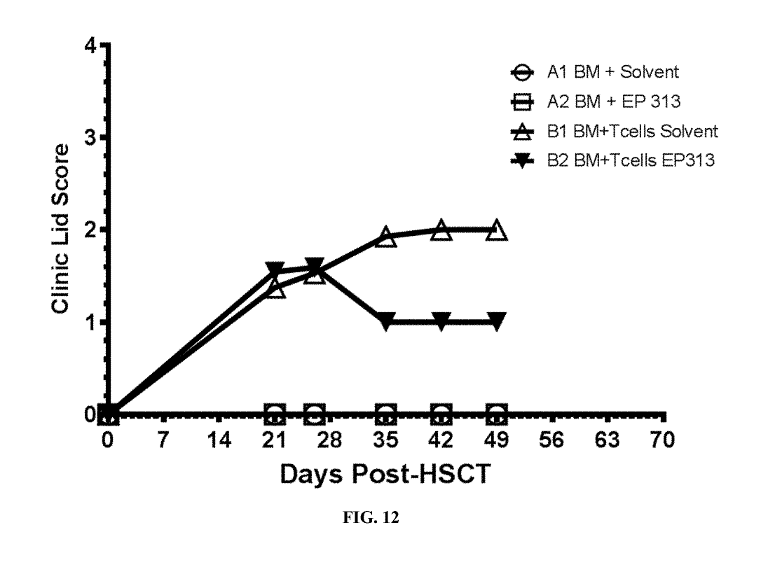

FIGS. 11A-11B show a dot plot representing individual eyelid scores and a line graph demonstrating the overall clinical response over the course of the experiment OGVHD #28, respectively. These findings indicated that local treatment with the BETi EP313 decreased inflammation of the lid as evidenced by decreased lid edema. Similarly FIG. 12 demonstrates the resolution of lid edema as seen using a different treatment protocol with EP313 (OGVHD #33; see FIG. 10). These data validate that there is an effect of the BETi being assessed, and suggest that modification of the treatment regimen may be able to increase efficacy.

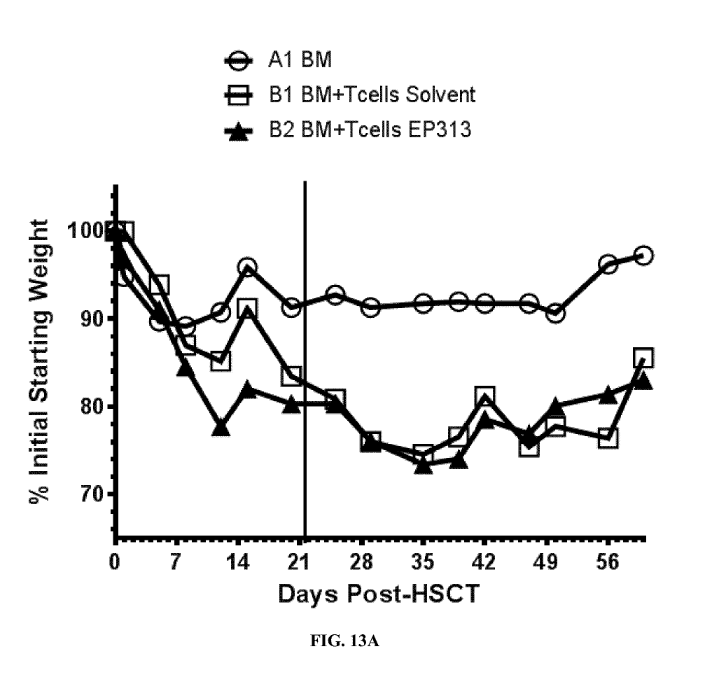

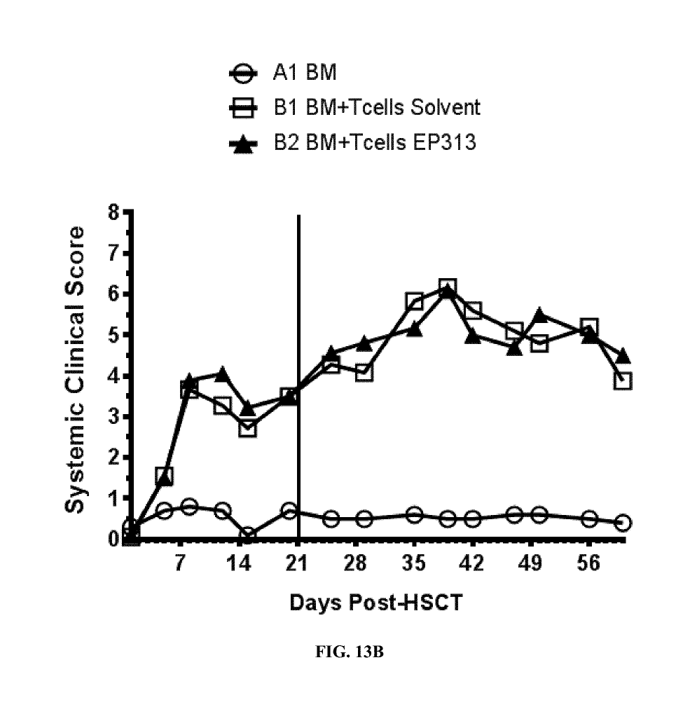

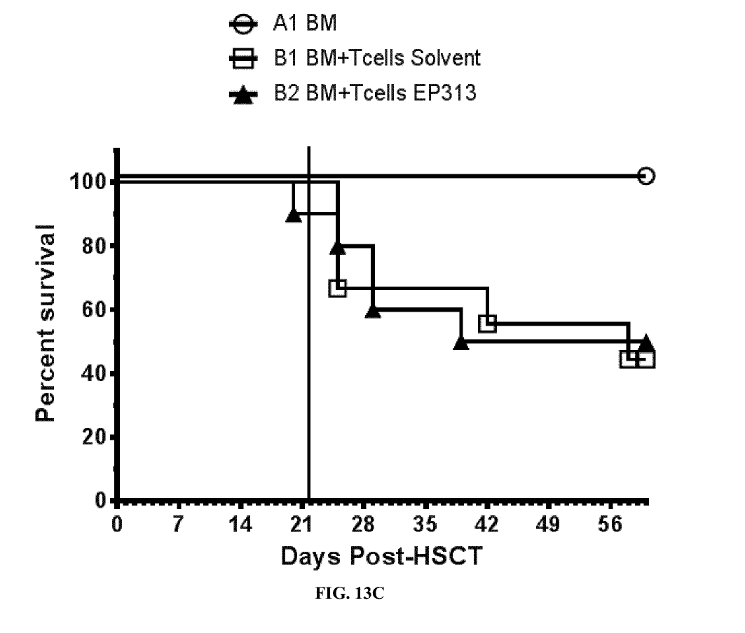

FIGS. 13A-13C illustrate the development of systemic GVHD in our MUD model of B6→C3H.SW as assessed by weight loss (FIG. 13A), clinical score (FIG. 13B), and overall survival (FIG. 13C). These data sets illustrate the progress of systemic GVHD in this mouse model, and demonstrate that the local administration of EP313 does not affect the development of this GVHD.

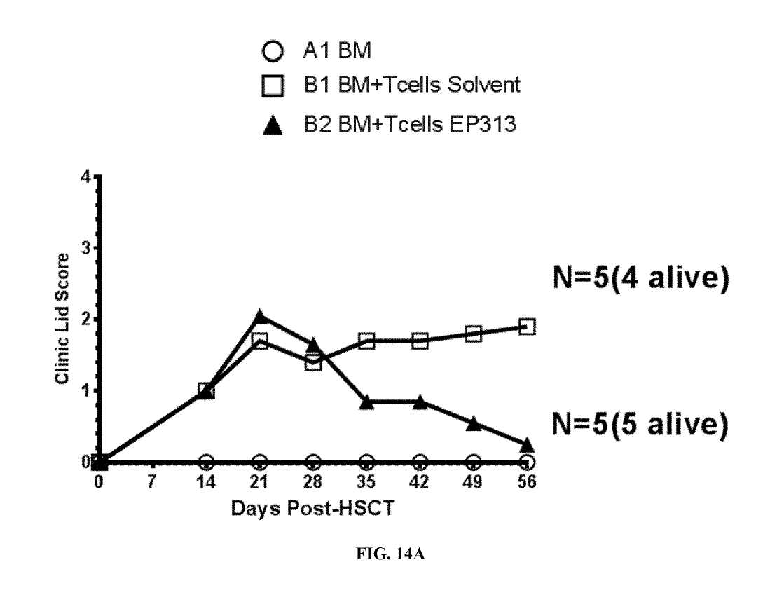

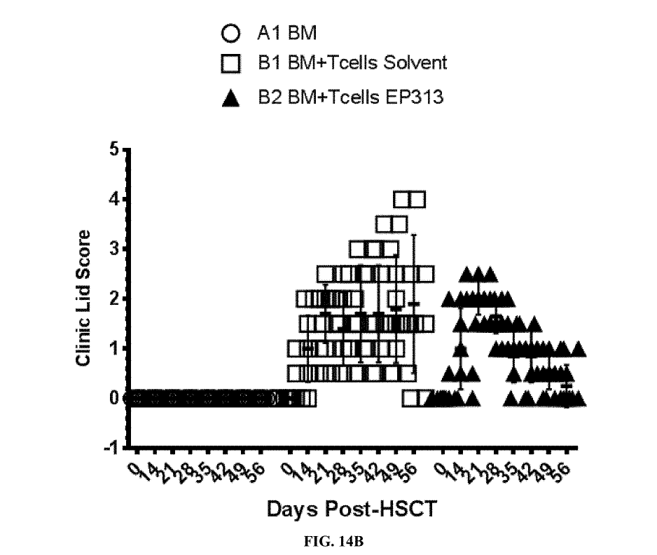

FIGS. 14A-14B (OGVHD #36) demonstrate the ocular clinical response to local application of EP313 in these mice with systemic ocular GVHD. In this experiment treatment was further modified to include topical and subconjunctival application (FIG. 10). The results demonstrate the significant resolution of lid edema over the course of treatment for 6 weeks which was initiated after GVHD onset (FIG. 14A). Additionally, FIG. 14B represents individual eyelid scores recorded throughout the duration of the experiment. It is important to note that as the experiment progressed, more animals died. As a result, the lid scores for these deceased animals do not appear at later times and as indicated, by Day 56, there were only 5 mice remaining alive in the EP313 treated group, and only 4 mice remaining alive in the solvent treated group. In total, the results from these three ocular GVHD experiments support the notion that ocular GVHD can be treated by local administration of bromodomain inhibitors which epigenetically regulate local inflammatory cytokines.

The data establish that local manipulation of the ocular infiltration and regulatory pathways associated with donor and potential host resident inflammatory cells in the ocular adnexa can be targeted to efficiently prevent and treat ophthalmic GVHD. The use of bromodomain inhibitors provides important advantages in regulating inflammation in the ocular compartment because of the ability to regulate gene transcription and cytokine/chemokine production by both infiltrating and local parenchymal cell populations. Combining bromodomain inhibitors with compounds that block recruitment of infiltrating cells is an attractive therapeutic option for controlling sterile inflammation associated with GVHD ocular damage.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Throughout the specification, where formulations are described as including components or materials, it is contemplated that the formulations can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. An ophthalmic formulation comprising a bromodomain inhibitor, a polar aprotic solvent, polysorbate, salt, and Captisol.

2. The formulation of claim 1 comprising 0.5%-5% bromodomain inhibitor, 0.1%-1% DMSO, 0.1%-1% polysorbate-80, 0.1%-1.5% sodium chloride, and 1%-2.5% Captisol.

3. The formulation of claim 1 comprising 0.5%-5% bromodomain inhibitor, 0.5% DMSO, 0.5% polysorbate-80, 0.9% sodium chloride, and 1.8% Captisol.

4. The formulation of any one of claims 1-3, wherein the bromodomain inhibitor is JQ1, IBET-151, or EP313.

5. A method of treating or preventing ophthalmic inflammation in a subject in need thereof, the method comprising locally administering the formulation of any one of claims 1-4 to the eye of the subject.

6. The method of claim 5, comprising topically administering the formulation to the surface of the eye.

7. The method of claim 5, comprising subretinally administering the formulation to the eye.

8. The method of any one of claims 5-7, wherein the ophthalmic inflammation is keratoconjunctivitis sicca, ocular surface inflammation, inflammation resulting from allergies, keratitis, conjunctivitis, uveitis, inflammation associated with diabetic retinopathy, inflammation associated with age related macular degeneration, cyclitis, scleritis, episcleritis, blepharitis, inflammation associated with corneal and/or conjunctival ulcer, inflammation associated with abrasion or wound to the eye, inflammation associated with ophthalmic graft versus host disease (GVHD), or inflammation associated with ophthalmologic surgery.

9. The method of claim 8, wherein the ophthalmic inflammation is keratoconjunctivitis sicca.

10. The method of claim 8, wherein the ophthalmic inflammation is inflammation associated with ophthalmic GVHD.

9. The method of claim 8, wherein the ophthalmic inflammation is keratoconjunctivitis sicca.

10. The method of claim 8, wherein the ophthalmic inflammation is inflammation associated with ophthalmic GVHD.
